(19) United States
(12) Patent Application Publication
(10) Pub. No.: US 2015/0282848 A1
(43) Pub. Date: Oct. 8, 2015

(54) KNEE BRACE FIXATION APPARATUS AND METHOD FOR APPLICATION THEREOF

(71) Applicant: Rahul Vaidya, Tecumseh (CA)

(72) Inventor: Rahul Vaidya, Tecumseh (CA)

(21) Appl. No.: 14/168,003

(22) Filed: Jan. 30, 2014

(51) Int. Cl. A61B 17/80

(52) U.S. Cl. CPC A61B 17/8057; A61B 17/8061

(57) ABSTRACT

An orthopaedic implant knee brace and surgical method for minimally invasive insertion of the orthopaedic implant knee hinge. The apparatus and method stabilizes the knee in patients after conventional fixation of distal femur and/or proximal tibia fractures. The orthopaedic implant knee brace allows the body weight of the patient to be offloaded from the knee joint to allow for weight bearing on the leg within days, rather than months, from the surgery.

### FIELD OF THE INVENTION

The present invention relates generally to orthopedic implants and minimally invasive methods for insertion thereof. Specifically the present invention relates to an orthopaedic implant knee brace and surgical method for minimally invasive insertion thereof.

### BACKGROUND OF THE INVENTION

Distal femur and/or proximal tibia fractures often occur in high energy injuries, such as from a car crash. The breaks may extend into the knee joint and may shatter the bone into many pieces. These injuries are amongst the most challenging fractures to treat.

FIGS. 1a and 1b show the conventional means to fix the fractures 19 in the distal femur regions. Bone screws 21 with or without bone plates 20 are typically used to fix these fractures 19. FIGS. 1c and 1d show the conventional means to fix the fractures 19 in the proximal tibia region. Bone screws 21 with or without bone plates 20 are typically used to fix these fractures 19 also. Fixation by these techniques may require as much as 3 months or more of healing before weight bearing can be done safely. During this time, the patient will need crutches or a walker to move around.

Thus, there is a need in the art for an apparatus and method for supporting the knee in patients after fixation of distal femur and/or proximal tibia fractures and allowing the offloading of the body weight from the knee joint to allow for weight bearing on the leg within days, rather than months, from the surgery.

### SUMMARY OF THE INVENTION

The present invention provides for an orthopaedic implant knee brace supporting the knee in patients after fixation of distal femur and/or proximal tibia fractures and allowing the offloading of the body weight from the knee joint to allow for weight bearing on the leg within days, rather than months, from the surgery. The orthopaedic implant knee brace may comprise two elongated plates, each of said elongated plates may have more than one affixation opening therein to accommodate affixation means passing through said affixation opening. One of said two elongated plates may be adapted to be placed subcutaneously, but supra-muscularly on the medial side of the patient's knee spanning from at least the distal femur to the proximal tibia. The other of said two elongated plates may be adapted to be placed subcutaneously, but supra-muscularly on the lateral side of the patient's knee spanning from at least the distal femur to the proximal tibia. The knee brace may also include at least four affixation means per elongated plate to affix the knee brace to the bones of the femur and tibia, at least two of said at least four affixation means passing through said more than one affixation opening in each end of each of said elongated plates.

The affixation openings may be threaded, said affixation means may be a screw and said screw may have a threaded head which may cooperate with said threading in said affixation openings. The affixation means may be a screw and said screw may have threading on the shaft only on the end thereof that will be inserted into the bone. One or both of the elongated plates may have offsets regions and/or irregular shape to provide for proper placement on the femur and tibia. The affixation means mat also be a threaded rod combined with nuts to anchor said elongated plates to said rods. The elongated plates and said attachment means may be formed from a material selected from the group consisting of titanium, stainless steel or a bio-compatible polymer material.

The knee brace may comprise a single elongated plate placed on either the medial or lateral said of said knee, the single elongated plate formed of two elongated subplates joined together by a connector plate. One of said subplates may be adapted to be placed subcutaneously, but supra-muscularly adjacent one of the distal femur or the proximal tibia. The other of said subplates may be contoured and adapted to be placed sub-muscularly adjacent the other of the distal femur or the proximal tibia. The connector plate may comprise a lockable hinge.

The knee brace may comprise a single elongated plate placed on either the medial or lateral said of said knee, said single elongated plate formed of two elongated subplates joined together by a connector plate. One of said subplates may be contoured and adapted to be placed sub-muscularly adjacent the distal femur. The other of said subplates may be contoured and adapted to be placed sub-muscularly adjacent the other of the proximal tibia. The connector plate may comprise a lockable hinge.

The surgical method for minimally invasive insertion of the orthopaedic implant knee brace may comprise providing an orthopaedic implant knee brace comprising two elongated plates, each of said elongated plates having more than one affixation opening therein to accommodate affixation means passing through said affixation opening; one of said two elongated plates adapted to be placed subcutaneously, but supra-muscularly on the medial side of the patient's knee spanning from at least the distal femur to the proximal tibia and the other of said two elongated plates adapted to be placed subcutaneously, but supra-muscularly on the lateral side of the patient's knee spanning from at least the distal femur to the proximal tibia. The method may further comprise the step of tunneling said orthopaedic implant knee brace subcutaneously in the subcutaneous fat layer parallel to the length dimension of the femur and tibia, one each along the lateral and medial side of the leg. The method may also comprise the step of attaching the ends of each elongated plate to the distal end of the femur and the proximal end of the tibia, wherein said orthopaedic implant knee brace remains disposed in the subcutaneous fat layer and away from, but parallel to the femur and tibia once attached thereto.

The tunneling step may include the further step of creating one or more incisions in the skin on both the lateral and medial side of the leg near both the distal end of the femur and the proximal end of the tibia, and said two elongate plates may be implanted, one on the lateral side and one on medial side of the knee.

The step of attaching the ends of the orthopaedic implant knee brace to the distal end of the femur and the proximal end of the tibia may further include the step of inserting at least four affixation means per elongated plate to affix the knee brace to the bones of the femur and tibia.

### BRIEF DESCRIPTION OF THE FIGURES

FIG. 1a depicts the conventional fixation of distal femur fractures using only bone screws;

FIG. 1b depicts the conventional fixation of distal femur fractures using bone screws and a bone plate;

FIG. 1c depicts the conventional fixation of proximal tibia fractures using only bone screws;

FIG. 1d depicts the conventional fixation of proximal tibia fractures using bone screws and a bone plate;

FIG. 2 is a depiction of a front view of a knee joint with the knee brace of the present invention attached thereto;

FIG. 3 is a depiction of a front view of the knee joint with another embodiment of the knee brace of the present invention attached thereto;

FIG. 4 is a depiction of the front view of the knee joint with yet another embodiment of the internal brace of the present invention attached thereto;

FIG. 5 is a depiction of the front view of the knee joint with still another embodiment of the internal brace of the present invention attached thereto;

FIG. 6 depicts a preferred affixation means, a screw, useful to affix the inventive knee hinge.

### DETAILED DESCRIPTION OF THE INVENTION

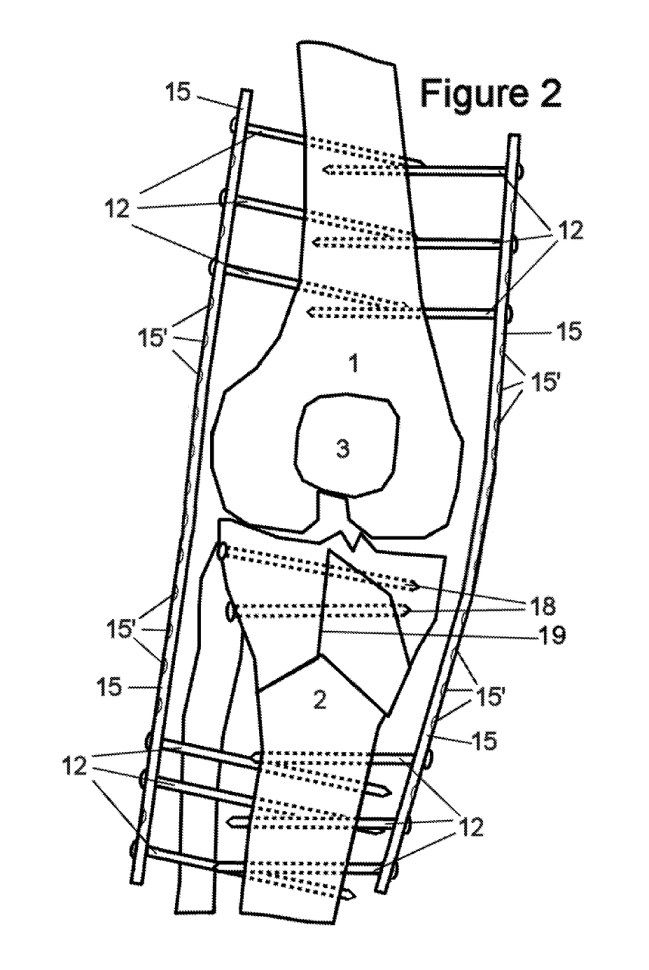

The present invention is a internal knee brace orthopedic implant device and method for application thereof. FIG. 2 is a depiction of a front view of a knee joint with the knee brace of the present invention attached thereto. The knee joint does not show and of the muscles, ligaments or tendons. In its most basic form the knee brace is composed of two elongated plates 15 one placed on either side of the knee joint with multiple affixation openings therein.

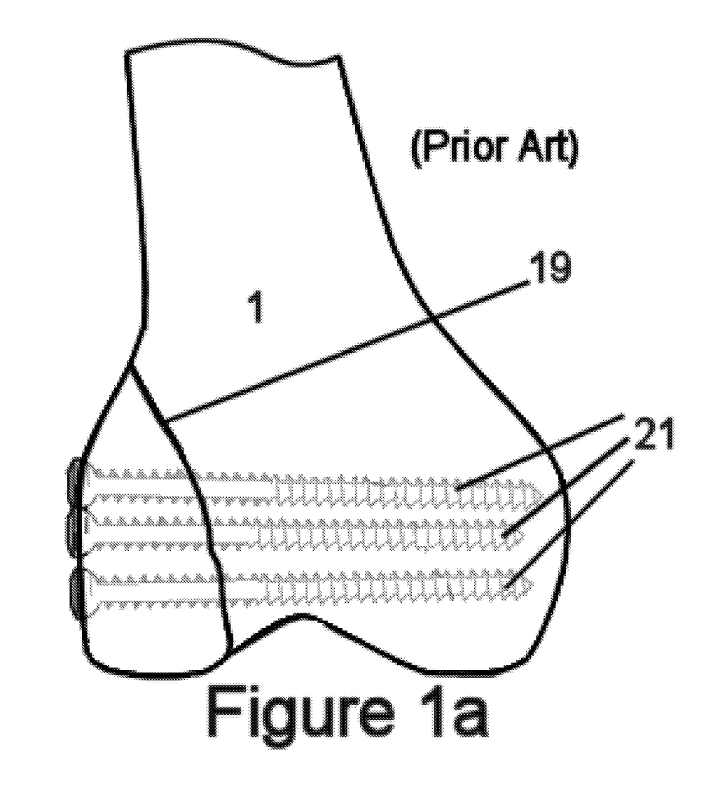

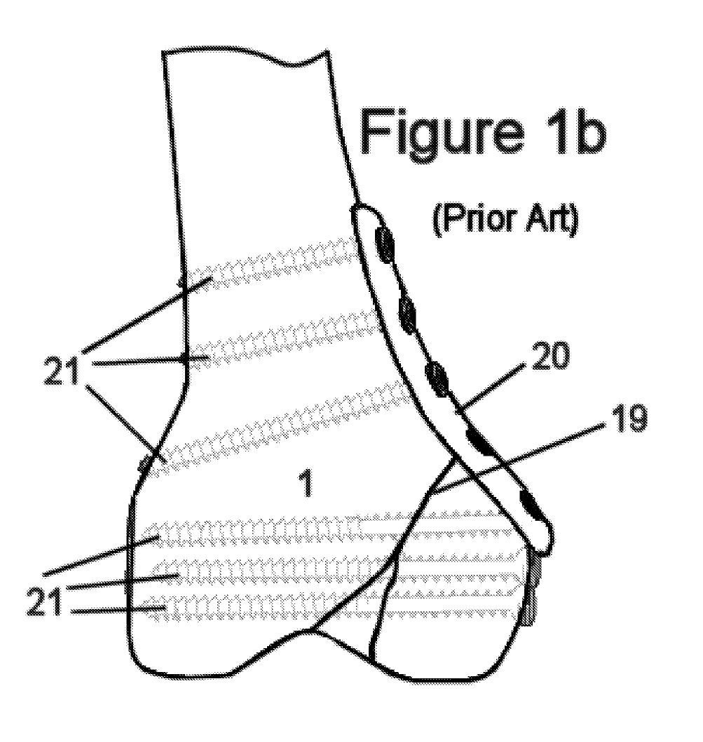

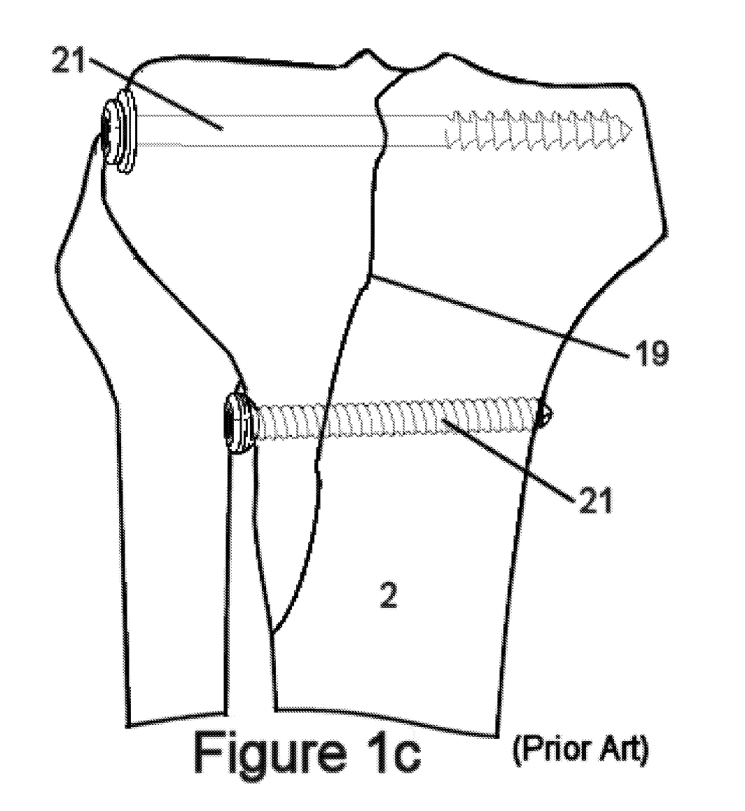

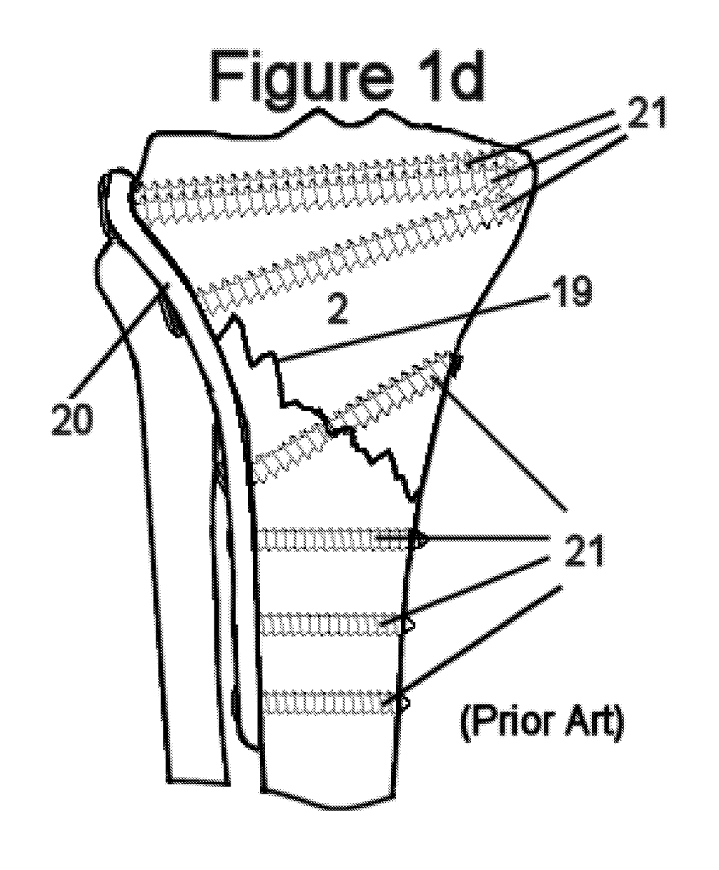

The two elongated plates 15 are attached to both the femur 1 and the tibia 2. The knee brace allows the weight of the body to be offloaded from the femur 1 above the injured knee to the tibia 2 below the injured knee, allowing the limb to bear weight without additional injury or stress to the knee joint. The two elongated plates 15 are connected to the tibia and femur using attachment means 12 which pass through the elongated plates 15 via affixation openings 15′ and into the femur 1 and tibia 2. If needed, one or both of the elongated plates may have offsets regions and irregular shape to provide for proper placement on the femur 1 and tibia 2. FIG. 2 shows the brace of the present invention used in conjunction with the conventional fixation of proximal tibia fractures using bone screws 21 as shown in FIG. 1c (the fixation could also have used a bone plate 20 as in FIG. 1d). In fact, the knee brace can be used in conjunction with conventional fixation of distal femur and/or proximal tibia fractures, including those shown in FIGS. 1a-1d and combinations thereof.

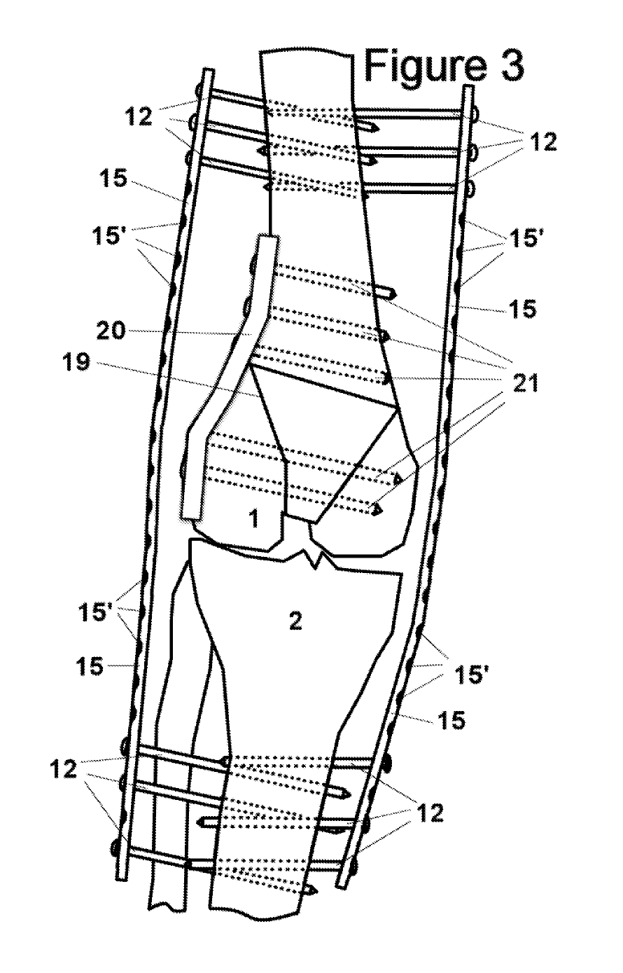

FIG. 3 is a depiction of the front view of the knee joint with the knee brace of the present invention attached thereto. Again the knee joint does not show any of the muscles, ligaments or tendons. It should be noted that the elongated plates of the knee hinge of the present invention are placed subcutaneously but supra-muscularly. This allows for proper stabilization of the knee without having to cut through the muscles and tendons to place the plates against the bones. In FIG. 3. the knee has fractures of the distal femur 19 which have been conventionally fixed as shown in FIG. 1b using bone screws 21 and a bone plate 20 (the fixation could also have used just bone screws 21 as in FIG. 1a). As in FIG. 2 above, the knee brace allows the weight of the body to be offloaded from the femur 1 above the injured knee to the tibia 2 below the injured knee, allowing the limb to bear weight without additional injury or stress to the knee joint.

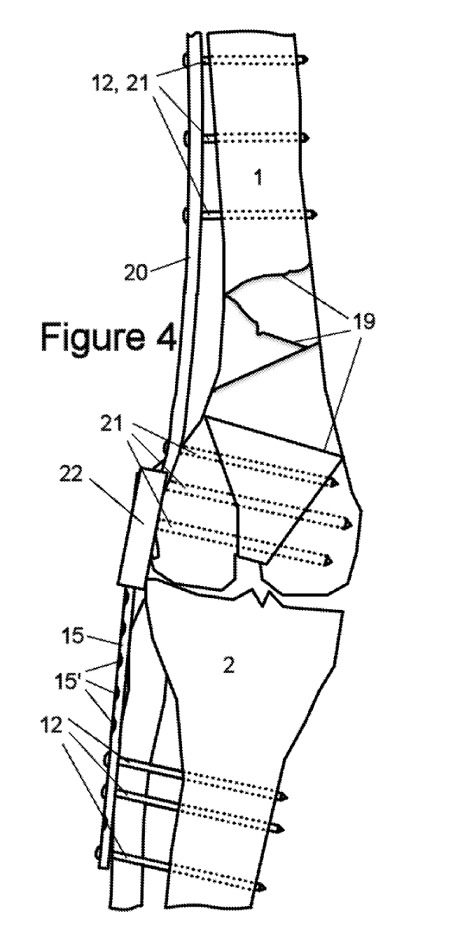

FIG. 4 is a depiction of the front view of the knee joint with another embodiment of the internal brace of the present invention attached thereto. Again the knee joint does not show any of the muscles, ligaments or tendons. The knee has fractures 19 of the distal femur which have been conventionally fixed as shown in FIG. 1b using bone screws 21 and a bone plate 20 as in FIG. 1b. However, unlike the conventional fixation technique, an additional plate 15 has been inserted subcutaneously along the proximal tibia and secured to the tibia vial attachment means 12. The additional plate 15 is attached to the conventional fixation plate 20 used on the femur by a connector plate 22. This connector plate allows for offloading of the body weight from the conventional fixation plate 20 through the connector plate 22 and into plate 15 which transfers the load to the tibia, once again allowing the limb to bear weight without additional injury or stress to the knee joint.

The connector plate may also take the form of a lockable hinge. This would allow the leg to bear weight when the hinge is locked and allow for bending of the knee (if appropriate) when the hinge is unlocked. This would allow the patient to retain range of motion in the knee joint from the beginning of the healing process and yet be able to bear weight on the limb.

It should be noted that the embodiment in FIG. 4 can also be used for injury to the proximal tibia such as shown in FIG. 1d where the conventional plate 20 would be attached to the tibia and the additional plate 15 would be attached subcutaneously (supra-muscularly) along the distal femur by attachment means 12. As with the other embodiment, the additional plate 15 is attached to the conventional fixation plate 20 used on the tibia by a connector plate 22. Once again, this connector plate allows for offloading of the body weight from the femur 1 to plate 15, through the connector plate 22 and into the conventional fixation plate 20 which transfers the load to the tibia.

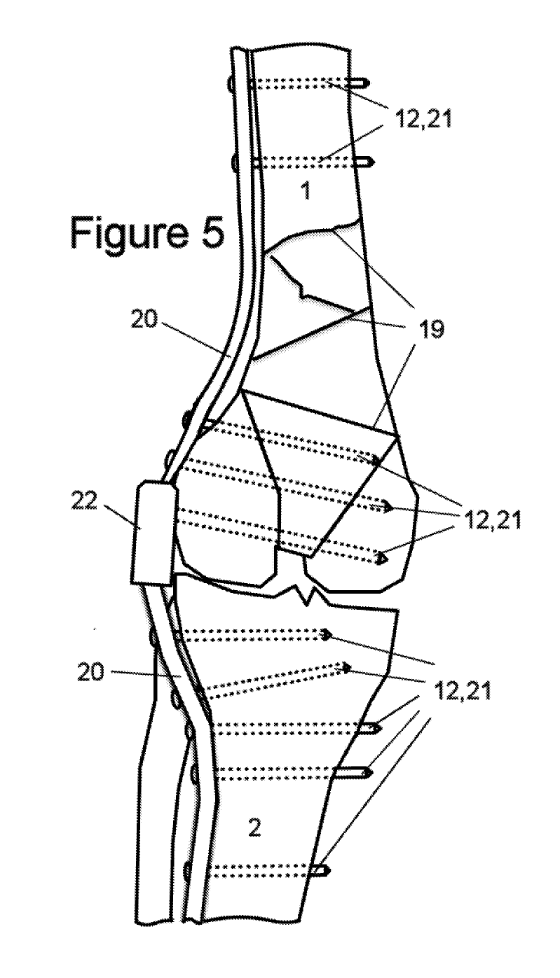

FIG. 5 is a depiction of the front view of the knee joint with yet another embodiment of the internal brace of the present invention attached thereto. As always, the knee joint does not show any of the muscles, ligaments or tendons. This embodiment can be used on the knee when there are distal femur fractures, proximal tibia fractures or both. The embodiment uses conventional screw 21 and plate 20 fixation of the fractures and screw 21 and plate 20 attachment to any non-fractured bone of the knee joint. Again, the knee has fractures 19 of the distal femur which have been conventionally fixed as shown in FIG. 1b using bone screws 21 and a bone plate 20 as in FIG. 1b. The unfractured tibia is fitted with a conventional screw 21 and plate 20 fixation implant. Then the conventional plate 20 on the femur and the conventional plate 20 on the tibia are connected together by a connector plate 22. This connector plate allows for offloading of the body weight from the conventional fixation plate 20 on the femur through the connector plate 22 and into the conventional plate 20 on the tibia, which transfers the load to the tibia, once again allowing the limb to bear weight without additional injury or stress to the knee joint. Again, the connector plate may take the form of a lockable hinge to provide the benefits described above.

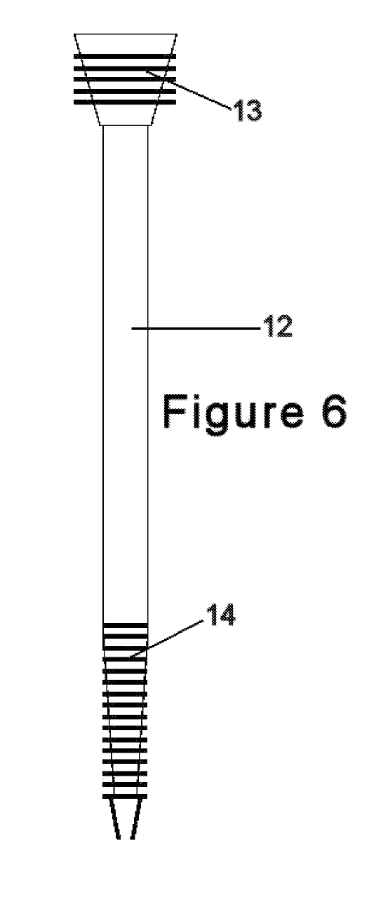

FIG. 6 depicts a preferred affixation means 12, a screw. The screw 12 may preferably have a threaded head 13 which may cooperate with threading in the affixation openings 15′ of the elongated plates 15. The affixation openings 15′ may be threaded as in locking plate technology. This feature allows the elongated plates 15 to remain in place subcutaneously but supra-muscularly without being pressed against the muscles, yet holding the bones firmly in place. The screw 12 also preferably has thread 14 only on the portion of the shaft thereof that will be inserted into the bone. Alternatively, a threaded rod may also be used to attach the plates to the bones using nuts or the like to anchor the plates to the rods in the subcutaneous position. The elongated plates 15, the attachment means 12 and the connector plate 22 may be formed from titanium, stainless steel or a bio-compatible polymer material.

The knee brace may be placed into the subcutaneous fat layer through two incisions in the skin. One incision is near the distal end of the femur 1 and the other is near the proximal end of the tibia 2. The incisions may be approximately two inches or less on each end. Of course, the plates 15 may come in many different sizes to accommodate different sized people and bones. This placement of the elongated plates 15 just under the skin prevents disruption of the muscle tissue and since there is no dissection, there is little chance for infection. It should be noted that the braces are not a permanent implant, but rather should be removed after the injury to knee joint has healed.

It is to be expected that considerable variations may be made in the embodiments disclosed herein without departing from the spirit and scope of this invention. Accordingly, the significant improvements offered by this invention are to be limited only by the scope of the following claims.

I claim:

1. A orthopaedic implant knee brace comprising:
two elongated plates, each of said elongated plates having more than one affixation opening therein to accommodate affixation means passing through said affixation opening;
one of said two elongated plates adapted to be placed subcutaneously, but supra-muscularly on the medial side of the patient's knee spanning from at least the distal femur to the proximal tibia and the other of said two elongated plates adapted to be placed subcutaneously, but supra-muscularly on the lateral side of the patient's knee spanning from at least the distal femur to the proximal tibia; and
at least four affixation means per elongated plate to affix the knee brace to the bones of the femur and tibia, at least two of said at least four affixation means passing through said more than one affixation opening in each end of each of said elongated plates.

2. The orthopaedic implant knee brace of claim 1, wherein said affixation openings are threaded, said affixation means is a screw and said screw has a threaded head which cooperates with said threading in said affixation openings.

3. The orthopaedic implant knee brace of claim 1, wherein said affixation means is a screw and said screw has threading on the shaft only on the end thereof that will be inserted into the bone.

4. The orthopaedic implant knee brace of claim 1, wherein one or both of the elongated plates has offsets regions and/or irregular shape to provide for proper placement on the femur and tibia.

5. The orthopaedic implant knee brace of claim 1, wherein said affixation means is a threaded rod combined with nuts to anchor said elongated plates to said rods.

6. The orthopaedic implant knee brace of claim 1, wherein said elongated plates and said attachment means are formed from a material selected from the group consisting of titanium, stainless steel or a bio-compatible polymer material.

7. The orthopaedic implant knee brace of claim 1, wherein said knee brace comprises a single elongated plate placed on either the medial or lateral said of said knee, said single elongated plate formed of two elongated subplates joined together by a connector plate;
one of said subplates is adapted to be placed subcutaneously, but supra-muscularly adjacent one of the distal femur or the proximal tibia;
the other of said subplates is contoured and adapted to be placed sub-muscularly adjacent the other of the distal femur or the proximal tibia.

8. The orthopaedic implant knee brace of claim 7, wherein said connector plate comprises a lockable hinge.

9. The orthopaedic implant knee brace of claim 1, wherein said knee brace comprises a single elongated plate placed on either the medial or lateral said of said knee, said single elongated plate formed of two elongated subplates joined together by a connector plate;
one of said subplates is contoured and adapted to be placed sub-muscularly adjacent the distal femur;
the other of said subplates is contoured and adapted to be placed sub-muscularly adjacent the other of the proximal tibia.

10. The orthopaedic implant knee brace of claim 9, wherein said connector plate comprises a lockable hinge.

11. A surgical method for minimally invasive insertion of an orthopaedic implant knee brace comprising the steps of:
providing an orthopaedic implant knee brace comprising two elongated plates, each of said elongated plates having more than one affixation opening therein to accommodate affixation means passing through said affixation opening; one of said two elongated plates adapted to be placed subcutaneously, but supra-muscularly on the medial side of the patient's knee spanning from at least the distal femur to the proximal tibia and the other of said two elongated plates adapted to be placed subcutaneously, but supra-muscularly on the lateral side of the patient's knee spanning from at least the distal femur to the proximal tibia;
tunneling said orthopaedic implant knee brace subcutaneously in the subcutaneous fat layer parallel to the length dimension of the femur and tibia one each along the lateral and medial side of the leg; and
attaching the ends of each elongated plate to the distal end of the femur and the proximal end of the tibia;
wherein said orthopaedic implant knee brace remains disposed in the subcutaneous fat layer and away from, but parallel to the femur and tibia once attached thereto.

12. The surgical method of claim 11, wherein said tunneling step includes the further step of creating one or more incisions in the skin on both the lateral and medial side of the leg near both the distal end of the femur and the proximal end of the tibia, and said two elongate plates are implanted, one on the lateral side and one on medial side of the knee.

13. The surgical method of claim 11, wherein said step of attaching the ends of the orthopaedic implant knee brace to the distal end of the femur and the proximal end of the tibia includes inserting at least four affixation means per elongated plate to affix the knee brace to the bones of the femur and tibia.

14. The surgical method of claim 13, wherein said affixation openings are threaded, said affixation means is a screw and said screw has a threaded head which cooperates with said threading in said affixation openings.

15. The surgical method of claim 13, wherein said affixation means is a screw and said screw has threading on the shaft only on the end thereof that will be inserted into the bone.

16. The surgical method of claim 13, wherein one or both of the elongated plates has offsets regions and/or irregular shape to provide for proper placement on the femur and tibia.

17. The surgical method of claim 13, wherein said affixation means is a threaded rod combined with nuts to anchor said elongated plates to said rods.

18. The surgical method of claim 13, wherein said elongated plates and said attachment means and are formed from a material selected from the group consisting of titanium, stainless steel or a bio-compatible polymer material.
